(19) United States
(12) Patent Application Publication
(10) Pub. No.: US 2010/0063461 A1
(43) Pub. Date: Mar. 11, 2010

(54) COMPACT LINEAR IMPLANTABLE SITE

(76) Inventor: Marc José Esteve, Paris (FR)

Correspondence Address: THOMAS, KAYDEN, HORSTEMEYER & RISLEY, LLP, 600 GALLERIA PARKWAY, S.E., STE 1500, ATLANTA, GA, 30339-5994, US

(21) Appl. No.: 12/375,800

(22) PCT Filed: Aug. 3, 2007
(86) PCT No.: PCT/FR07/01341

§ 371 Date: Sep. 8, 2009

(30) Foreign Application Priority Data
Aug. 3, 2006 (FR) 0607137

(51) Int. Cl. A61M 31/00

(52) U.S. Cl. 604/288.02

(57) ABSTRACT

A device intended to be surgically introduced under the skin of a human or animal patient and capable of being subsequently pierced by a hollow needle, through the skin of the patient, for the purpose of introducing and/or drawing substances into or from the body of the patient. The device may include an outlet capable of connecting the implantable device with a duct such as a catheter.

### TECHNICAL FIELD OF THE INVENTION

The present invention relates to the general technical field of devices intended to be surgically introduced under the skin of a human or animal patient so as to be capable of being pierced subsequently by a hollow needle, through the skin of the patient, for the purpose of introducing and/or drawing substances into or from the body of the patient, while limiting trauma, notably of the skin, linked to repeated pricks.

Such devices are generally referred to as “implantable sites”.

More particularly, the present invention relates to an implantable device for injecting and/or drawing fluid into or from a human or animal organism, said device comprising a chamber extending between a puncture area, adapted to be capable of being pierced by a needle for the purpose of injecting and/or drawing a fluid, and an outlet located opposite said puncture area and intended to connect said chamber with a duct such as a catheter, said chamber being so shaped to receive the needle.

The present invention also relates to a system comprising an implantable device in accordance to the previous paragraph.

### PRIOR ART OF THE INVENTION

It is known to use implantable devices to provide a direct parenteral access to any part of the body (organ, vessel, cavity . . . ) for the purpose of performing food and drug substance administration, blood drawing, or else drainage works, on an iterative basis.

Generally, for this purpose, a system is used which consists of a elastomeric tubing connected, at one of its ends, to an implantable site in the form of an hermetically tight housing comprising at least one chamber for receiving the fluid that is punctured or injected. Said chamber is closed by means of a self-healing membrane, or “septum”, which forms a puncture area capable of being pierced several times by a hollow needle without losing its tightness.

Commonly, the site is implanted under the skin of the patient, while the second end of the elastomeric tubing is placed in the organ, cavity or vessel into or from which a fluid has to be diffused or drawn. Thus, the practitioner can reach said organ through an offset access port. The cutaneous barrier surrounding the site advantageously limits the risks of infection linked to repeated pricks.

Nevertheless, the known implantable sites suffer from non-negligible drawbacks.

Firstly, the useful surface area of the puncture area is generally reduced in comparison with the whole bulkiness of the site.

Because of the smallness of this useful surface area, it is often difficult for the practitioner to locate the puncture point, which is a source of discomfort and pain for the patient since the puncture is badly performed or carried out only after several fruitless attempts.

Further, the substantial bulkiness of the site is in itself physically and aesthetically inconvenient because the patient's skin is visibly deformed by the implant.

Moreover, the arrangement of prior art sites and the compactness of the space available for approaching and maneuvering the needle often lead the practitioner to bump the bevel of the needle into a rigid obstacle inside the site, such as a metallic wall. Now, if the bevel of the needle is twisted or warped, for example, when the needle firmly or brutally enters in contact with a hard obstacle, it is liable to involve degradation of the septum at the time the needle is extracted, and thus to prematurely compromise the tightness of the site.

Finally, the prior art implantable sites are generally made up of a large number of mechanical parts, so that they are complex to assemble and tend to have an unnecessary bulkiness while keeping a high fabrication cost.

### SUMMARY OF THE INVENTION

Consequently, the objects assigned to the invention are to remedy the various above-mentioned drawbacks and to provide a novel implantable device for injecting and/or drawing fluid into or from an organism, having a particularly simple and compact design while ensuring reliability and safety of operation.

Another object of the invention is to provide a novel implantable device that causes only minimal physical or aesthetical inconvenient for the patient.

Another object of the invention is to provide a novel implantable device having an optimized cost price.

Another object of the invention is to provide a novel implantable device offering good ergonomics and easy to implement by the practitioner, both at the time of implantation and during use, and in particular, easy to locate after implantation and allowing the practitioner to prick in a natural way.

The objects assigned to the invention are also to propose a novel system comprising an implantable device for injecting and/or drawing fluid into or from an organism, being particularly simple, compact and safe to use.

Finally, the objects assigned to the invention are to propose a novel system comprising an implantable device for injecting and/or drawing fluid into or from an organism, having an optimized service life.

The objects assigned to the invention are achieved by means of an implantable device for injecting and/or drawing fluid into or from a human or animal organism, said device comprising a chamber extending between a puncture area, adapted to be capable of being pierced by a needle for the purpose of injecting and/or drawing a fluid, and an outlet located opposite said puncture area and intended to connect said chamber with a duct such as a catheter, said chamber being so shaped to receive the needle, said device being characterized in that it has a substantially elongated shape along a longitudinal axis, in that the puncture area, the chamber and the outlet are substantially aligned in the direction of said longitudinal axis, and in that it comprises an interposing means projecting into the chamber so as to prevent the needle that enters said chamber through the puncture area from reaching the outlet.

The objects assigned to the invention are also achieved by means of a system comprising an implantable device according to the invention and having a catheter connected to said device so that the chamber communicates with said catheter through the outlet.

### BRIEF SUMMARY OF THE DRAWINGS

Other features and advantages of the invention will appear in greater detail from the following description, with reference to the appended drawings given only by way of illustrative and non-limiting example, in which:

FIG. 1 is a longitudinal cross-sectional view of a first embodiment variant of a device according to the invention;

FIG. 2 is an A-A cross-sectional view of the device of FIG. 1;

FIG. 3 is a longitudinal cross-sectional view of a second embodiment variant of a device according to the invention;

FIG. 4 is a B-B cross-sectional view of the device of FIG. 3;

FIGS. 5a and 5b are cut-away partial perspective views of an embodiment variant of interposing means capable of being implemented inside a device according to the invention, before and after contact of the needle with said interposing means, respectively;

FIG. 6 is a longitudinal cross-sectional view of a third embodiment variant of a device according to the invention;

FIG. 7 is a C-C cross-sectional view of the device of FIG. 6;

FIG. 8 is a D-D cross-sectional view of the device of FIG. 6;

FIG. 9 is a longitudinal cross-sectional view of a fourth embodiment variant of a device according to the invention;

FIG. 10 is a longitudinal cross-sectional view of a fifth embodiment variant of a device according to the invention;

FIG. 11 is a longitudinal cross-sectional view of a sixth embodiment variant of a device according to the invention;

FIG. 12 is an E-E cross-sectional view of the device of FIG. 11;

FIG. 13 is a longitudinal cross-sectional view of a seventh embodiment variant of a device according to the invention;

FIG. 14 is a G-G cross-sectional view of the device of FIG. 13;

FIG. 15 is a cut-away perspective view of an eighth embodiment variant of a device according to the invention;

FIG. 16 is a perspective view of a variant of a system according to the invention comprising a device according to the invention to which is connected a catheter;

FIG. 17 is a partially cross-sectioned overall view of another variant of a system according to the invention comprising a device according to the invention to which is connected a catheter.

### BEST WAY OF IMPLEMENTING THE INVENTION

The present invention relates an implantable device 1 for injecting and/or drawing fluid into or from a human or animal organism.

Such a device 1, also referred to as “implantable site”, is intended to be surgically implanted into the body of a patient, preferably under the skin of the patient, to create an access point for introducing or extracting fluid substances into or from the body of said patient.

The implantable device 1 according to the invention may be implemented and adapted for different purposes.

Firstly, the implantable device 1 according to the invention may be designed for injecting and/or drawing fluid into or from an organ or a vessel of the body of a patient, and in particular the venous or arterial system of said patient, for example to permit injection of drug substances into a vein or an artery. According to a particular variant of this application, said device 1 may be adapted to form an artificial vein or artery the practitioner can prick through the skin, just like a natural vein, so as to inject a therapeutic substance or to take blood.

The device 1 according to the invention may also be adapted to feed implanted reservoirs associated to insulin or analgesic pumps, for example.

The implantable device 1 according to the invention may also be adapted for injecting or puncturing fluid into or from the inflatable or deflatable compartment of a surgical implant, such as an artificial sphincter, a balloon, or a gastric ring for constricting the stomach to treat obesity.

Below, reference is made more particularly for the device 1 according to the invention as a hypodermic device, namely a device intended to be positioned just under the skin of the patient.

However, said device 1 could also be implanted at other locations within the body of the patient, and in particular at a greater depth, without departing from the scope of the invention.

According to the invention, the implantable device 1 comprises a chamber 2 extending between a puncture area 3 and an outlet 4 located opposite said puncture area 3.

The puncture area 3 is adapted to be capable of being pierced by a needle 5, notably a hollow needle, for the purpose of injecting and/or drawing a fluid into or from the chamber 2.

Preferably, said puncture area 3 comprises a self-sealing membrane 10 or “septum” adapted to ensure tightness of the device 1 when it is pricked by the needle 5 and after said needle 5 is removed, wherein the orifice created when said membrane 10 is pierced by the needle 5 closes automatically after said needle is extracted.

This self-healing function may advantageously be obtained by the self-sealing membrane 10 being made of an elastomeric material, such as silicone, and being preferably prestressed in compression.

The outlet 4 is advantageously intended to connect the chamber 2 with a flexible or rigid duct, such as a catheter 6. Thus, the chamber 2 can open into the environment of the device 1 through the outlet 4.

Of course, the chamber 2 is so shaped to receive the needle 5. Within the meaning of the invention, “chamber” designates a region of the space, the volume and dimensions of which are sufficient for the needle 5 issuing from the puncture area 3 to be capable to pass through said region of the space and/or to reach almost all points of said region of the space.

In other words, the chamber 2 is arranged so as to permit functional introduction and progression of the needle 5 within the chamber and not to hinder fluid circulation between the chamber 2 and the reservoir of the injection device (such as a syringe) associated with said needle 5.

According to an important characteristic of the invention, the device 1 comprises an interposing means 12 projecting into the chamber 2 so as to prevent the needle 5 that enters said chamber 2 through the puncture area 3 from reaching, and a fortiori passing through, the outlet 4.

In other words, in a chamber 2 according to the invention but “naked”, i.e. without interposing means 12, it would exist at least one sufficiently clear passageway enabling a trajectory to be drawn that connects directly one point of the puncture area 3 to the outlet 4, trajectory along which a needle 5 entering through the puncture area 3 would be able to reach, or even pass through, the outlet 4.

Advantageously, this arrangement makes it possible to provide the device 1 with a simple and compact shape, by allowing the puncture area 3 to be placed near the outlet 4 while ensuring safe operation of said device despite this proximity.

Indeed, thanks to the interposing means 12 which stands between the puncture area 3 and the outlet 4, it is possible to bring these elements closer to each other without exposing the outlet 4, or the surroundings thereof, or a fortiori the catheter 6, to aggressions by the bevel of the needle 5 which would otherwise be liable to cause perforation, laceration or abrasion damages.

Reciprocally, this arrangement is also liable, as it will be described in detail hereinafter, to limit the risks of damaging the bevel of the needle 5 when it contacts the implantable site 1, and consequently to reduce the risk for the self-sealing membrane 10 to be torn by a damaged, notably warped or twisted, bevel when the needle 5 is extracted. The safe operation and the longevity of the device 1 are thus improved.

Particularly preferentially, the interposing means 12 is of course arranged so as not to risk compromising the first function of the device 1, which is to permit fluid circulation between the needle 5 and the chamber 2 on the one hand, and between the chamber 2 and the catheter 6 on the other hand.

More particularly, the space left for the needle 5 by the interposing means 12 is preferably compatible with introduction of at least all the useful part of the hollow needle 5, i.e. generally the beveled part thereof, from various pricking points and incidence angles with respect to the puncture area 3.

Moreover, the residual space of the chamber 2, that is available at the level of the interposing means 12 for the flowing of injected or punctured fluids, has preferably dimensions, notably in cross-section, at least equal to, and preferably greater than, those of the outlet 4.

According to the invention, the chamber 2 is delimited by a wall 7 wherein, preferably, said wall connects the puncture area 3 to the edge of the outlet 4. Said wall 7 may naturally have varied dimensions and geometries, without departing from the scope of the invention.

Particularly preferentially, the wall 7 is adapted to resist to perforation by the needle 5, at least in the space of the chamber 2 that is within reach of said needle 5.

More precisely, the wall 7 could be reinforced so as to resist to the mechanical action exerted by the bevel of the needle 5, at least in all the areas the interposing means 12 allows to be reached from the puncture area 3. Tightness of said wall 7, and consequently safe operation of the device 1, are thus ensured.

According to an embodiment variant, the wall 7 could comprise an elastomeric housing 17 of the silicone type, wherein said elastomeric housing 17 may be reinforced, at least partially and notably in the area that can be reached by the bevel of the needle 5, by means of a lattice.

Protection against perforation and/or tearing could also be provided by a shell. The materials used to prevent perforation of the silicone housing could be in the form of plates, platelets, or wires. And preferably, they will be chosen from titanium, stainless steel, or biocompatible polymers, such as, for example, polyetheretherketone (PEEK), polysulfone (PSU), polycarbonate (PC) or polyetheramide (PEI).

Moreover, while it is possible for said wall 7 to be entirely flexible and/or deformable, the latter will be preferably at least partially rigid or semi-rigid, to make it easier to grasp the device 1 through the patient's skin and to holding it for pricking the puncture area. To that end, the wall 7 may comprise a rigid frame, at least on a part thereof, or may have a rigid shell.

Advantageously, the same materials and the same elements can be used to stiffen all or a part of the structure of the device 1 and to provide the wall 7 with a resistance to aggressions by the needle (perforation, laceration or abrasion).

In particular, a shell in titanium, stainless steel, PEEK, PSU, PC or PEI, could be used to that end.

Preferably, the device 1, and more particularly the chamber 2, has a substantially elongated shape along a longitudinal axis (XX′).

Particularly preferably, the puncture area 3, the chamber 2 and the outlet 4 are substantially aligned in the direction of said longitudinal axis (XX′).

Preferably, the ratio between the length of the device 1, measured along the longitudinal axis (XX′) between the puncture area 3 and the outlet 4, and the whole transversal dimension of said device 1, and more particularly of the puncture area, is substantially comprised between 1.5 and 2. Thus, by way of example, the length of the device 1 may be substantially comprised between 15 mm and 20 mm, and its maximal width of the order of 10 mm.

Advantageously, such an elongated shape provides the device 1 with an atraumatic and discrete characteristic.

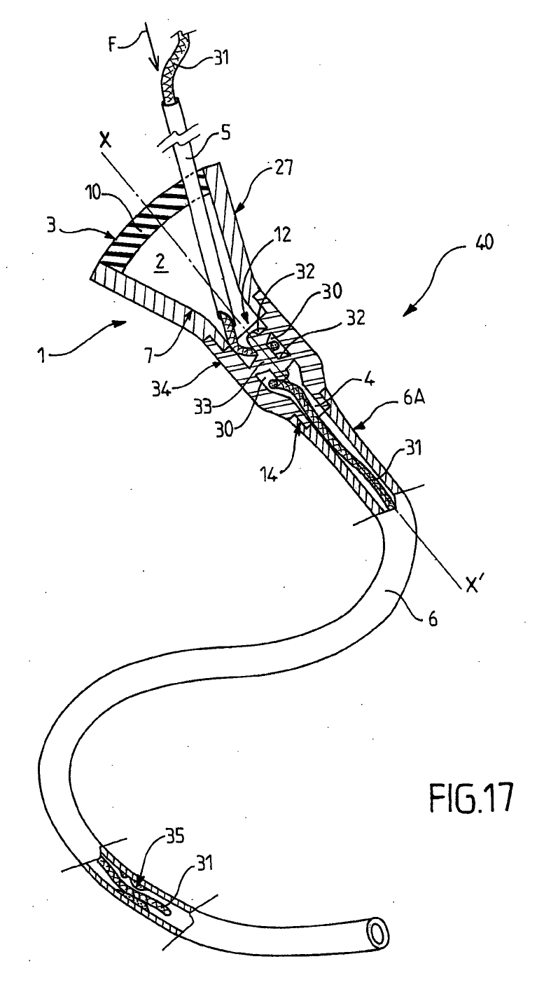

According to a preferential embodiment variant, the device 1 is shaped so that it can be connected in line with a catheter 6, as illustrated in FIGS. 16 and 17, the puncture area 3, the chamber 2, the outlet 4 and the end 6A of the catheter 6 that is adjacent to said outlet 4 being then preferably substantially aligned along the longitudinal axis (XX′). Preferably, at rest, the septum 10, the chamber 2 and the catheter 6 are substantially coaxial to each other.

Notably, thanks to such an arrangement in line, bulky or complex-shaped pieces for building a duct connecting the chamber 2 to the catheter 6 are not necessary. In particular, the use of intermediate ducts of a uselessly great length and/or a narrow cross-section, which present increased risks of obstruction, can thus be avoided.

Further, the combination of an interposing means 12 with an elongated shape ensures a good accessibility to the chamber, while saving a sufficient operating volume, a good circulation of fluids thanks to a suitably sized orifice, and a safe operation of the device without any early damage of the septum, the orifice or the catheter to which it is connected.

The device 1 may have an extension, preferably along the axis (XX′), forming a tip 14 for connection to the catheter 6. The length of the tip 14, comprising possibly a shoulder, advantageously permits the end 6A of the catheter 6 to be fixed thereto, notably by nesting, crimping and/or bonding.

According to an embodiment variant, the tip 14 can be reinforced so as to stiffen the end 6A of the catheter 6, in order to limit the risks of stenosis or degradation of said catheter 6 through kinking. In other words, the device 1 may be extended by a rigid or semi-rigid tip 14 which ensures continuity of the chamber 2 and which permits the end 6A of the catheter 6 to be fixed thereto, or even protected.

However, it can be noticed that, according to the invention, the outlet 4 and the catheter end 6A, though they are located opposite the puncture area 3 through which the needle 5 enters, are protected by the interposing means 12, whereby it not absolutely necessary to intrinsically reinforce them against aggressions (perforation, laceration, abrasion).

Thus, it is possible for the parts of the wall 7 that can not be reached by the needle 5 and/or the catheter 6 (including the end 6A thereof) to be made of simple-composition and cheap elastomeric materials, such as silicone or polyurethane (PU), and thus to limit the use of reinforced structures, notably composite or metallic ones, to the only parts of the device that are exposed to the bevel of the needle 5.

Particularly preferentially, only the fraction of the wall 7 that delimits the space of the chamber 2 within the reach of the needle 5 is both stiffened and reinforced against perforations, preferably by means of a single shell element, the rest of the wall 7 being made of an elastomeric material such as silicone or PU.

The above-mentioned elastomeric materials combining flexibility, structural simplicity, implementability and low cost, the device 1 according to the invention can thus present, in the one hand, a appreciable comfort of use because of the limited extent of the rigid areas, and on the other hand, a reduced fabrication cost.

According to a preferential embodiment variant, the device 1 according to the invention has a rotational geometry about the axis (XX′).

More preferentially, said device 1 has an external profile which flares, preferably in a continuous manner, toward the puncture area 3. It is possible for said profile to be bulged.

Advantageously, the combination of a linear arrangement with a rotational geometry provides the device 1 according to the invention with a compact, simple and substantially atraumatic shape. In particular, the shape-continuity offers a little hold to tissue coating. Moreover, the rotational symmetry of such a device 1 advantageously allows to avoid the problems linked to angular direction of the puncture area 3 with respect to the skin, the use of the implanted device being not affected by a possible turning of the latter on itself.

Even more preferentially, the device 1 has the form of a truncated cone 15 whose base 15A substantially corresponds to the puncture area 3 and apex 15B substantially corresponds to the outlet 4, as notably illustrated in FIGS. 1, 3, 5a, 5b, 6, 9, 10, 11, 13, 15 or 16. Of course, the base of said cone may have any shape, notably a circular, semi-circular, elliptical, polygonal shape. However, preferably, a base 15A and an apex 15B, i.e. a puncture area 3 and an outlet 4, respectively, substantially circular in shape and coaxial to each other are preferred.

Preferably, the height of the truncated cone 15, measured between the base 15A and the apex 15B, is substantially equal to 1.5 to 2 times the diameter of said base 15A. By way of example, the height of the truncated cone 15 may be substantially comprised between 15 mm and 20 mm, the diameter of said base 15A being substantially equal to 10 mm.

Advantageously, a wholly truncated shape for the device 1 facilitates the implantation thereof because the angular opening of the cone contributes to progressively separate the receiving tissues, for example the subcutaneous tissues, through corner effect when the device is introduced.

Further, a truncated shape for the device 1 allows a very easily identification and locating of the puncture area 3 by the practitioner, by simple palpation of the skin near the implantation area. Indeed, if the septum 10 is located at the base 15A, and covers preferably substantially the total extent of the latter, the locating thereof can be made by simple tactile and/or visual localization of the edge, preferably rounded, which marks the transition between said base 15A and the side of the truncated cone 15.

Moreover, the progressive separation offers a possibility to grasp the device 1 by pinching it through the skin and thus allows a certain manipulation by the practitioner, notably a dynamic angular rotation with respect to the hollow needle to make the introduction of the latter easier. According to a not shown embodiment variant, the external surface of the device 1 may be provided with means facilitating this transcutaneous grasping, such as, notably, concave bulged cavities intended to fit the terminal phalanxes of the thumb and the index finger.

According to an embodiment variant, the chamber 2 has also a flared shape, said chamber widening, preferably progressively, between the outlet 4 and the puncture area 3.

According to a preferential embodiment variant, the chamber 2 has a shape substantially mating the flared external profile of the device 1, notably a wholly truncated shape, such as illustrated in FIGS. 1, 3, 5a, 5b, 6, 9, 10, 11, 13, 15 and 16. The thickness of the wall 7 is then liable to be substantially constant.

In a particularly advantageous manner, the use of an implantable site 1 which flares toward the puncture area 3, a little as a funnel, allows to optimize accessibility to said site while maximizing the useful area for the penetration of a needle, i.e. notably the surface area of the self-sealing membrane 10, without having to significantly increase the whole volume, or the whole bulk of said implantable site 1.

In particular, the angular opening of the truncated cone 15, i.e. the solid angle formed by the chamber 2 “seen” from the outlet 4, allows varied approach angles of the needle when pricking. In other words, the implantable site 1 according to the invention having such an arrangement is particularly tolerant toward the pricking gesture. Thanks to the combination of the concinnity of the device 1 with the longitudinal arrangement thereof with respect to the catheter 6, the practitioner can advantageously prick substantially tangentially to the skin, which corresponds to the natural gesture of injection or puncturing into or from a vein.

According to a preferential embodiment variant of the invention, the interposing means 12 comprises at least one fixed element with respect to the wall 7 and/or with respect to the outlet 4 and/or with respect to the puncture area 3.

By “fixed element”, it is meant an element that is possibly flexible or deformable, but which has an attaching point that stands a substantially constant position with respect to the outlet 4, the wall 7 and/or the self-sealing membrane 10.

According to a preferential embodiment variant, the interposing means 12 may comprise one or more excrescencies 16 projecting from the wall 7 toward the inside of the chamber 2.

Particularly preferentially, said excrescencies are formed as a single-piece with the wall 7, and for example they are molded integral with said wall 7.

According to an embodiment variant, the interposing means 12 may comprise at least a plate 18 forming a baffle.

Preferably, said plate 18 extends in a plane cutting the axis (XX′) and, according to a particularly preferential embodiment variant, in a plane substantially perpendicular to the axis (XX′).

According to an embodiment variant, the interposition means 12 will comprise a plurality of plates 18, 19 forming baffle.

According to a preferential embodiment variant, said plates 18, 19 are stepped in baffle along the longitudinal axis (XX′), and preferably directed substantially perpendicular to said axis (XX′), as shown in FIG. 1.

Preferably, the residual space allowing the injected or punctured fluid to flow at the level of the baffle, and notably the surface defined between the ends of the plates 18, 19 and the wall 7 or else by the spacing between the plates themselves, is substantially greater than the surface area of the outlet 4, so as not to obstruct said flowing.

According to another embodiment variant illustrated in FIG. 10, the plates 18, 19 may form a side baffle sheltering the outlet 4. The so-delimited space for fluid flowing is then slightly offset with respect to the axis (XX′).

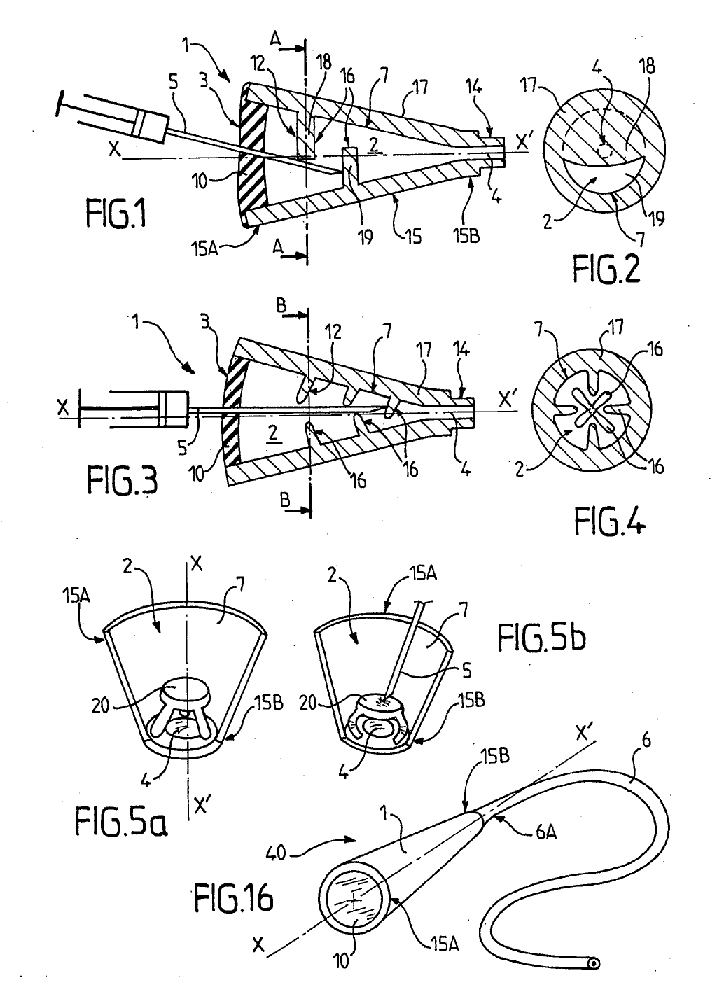

As illustrated in FIGS. 1, 2 and 10, said plates 18, 19 advantageously overlap each other so that the respective projections thereof onto a plane perpendicular to the longitudinal axis (XX′) cut together. In other words, the plates 18, 19 are preferably arranged so as to form a baffle hiding the solid angle corresponding to the angular opening of the chamber 2, and/or the puncture area 3, “seen” from the outlet 4.

Thus, whatever the penetration point of the needle 5 on the membrane 10, and whatever the penetration angle of said needle 5 into the chamber 2, said needle 5 can not reach directly the outlet 4 and necessarily meets on its way one or more plates 18, 19.

Noticeably, the interposing means 12, and more particularly the plates 18, 19, can then form either direct stops against the progression of the needle 5 within the chamber 2, or baffles tending to divert the trajectory of the needle 5 toward an element resisting to the perforation, such as the wall 7 or another constitutive element of the interposing means 12.

Moreover, the interposing means 12 can advantageously comprise flexible elements capable of mechanically resisting to the progression of the needle, without damaging the bevel of said needle 5.

In particular, the plates 18, 19 could be made of a reinforced elastomeric material adapted to support some elastic deformations and to resist to perforation without applying sever stress on the bevel of the needle 5.

It is also possible for the excrescencies 16 to be formed by a plurality of lugs attached to the wall 7 at their base, as shown in FIGS. 3 and 4.

According to an embodiment variant, it is possible that the interposing means 12 comprises a damping element 20 forming an elastic stop for the bevel of the needle 5, said damping element 20 being capable of deforming so as to progressively block the progression of said needle 5.

More particularly, such a damping element 20 may have the form of a shield or a net forming a screen between the chamber 2 and the outlet 4. Preferably, said screen can be supported by one or more flexible legs resting on the perimeter of the outlet 4 and thus form a kind of mushroom stop that can be crushed by the compression action of the bevel of the needle 5, as illustrated in FIGS. 5a and 5b.

Such a solution would notably allow to put a progressive resistance to introduction of the needle 5 within the chamber 2, so as to provide the practitioner with a tactile feeling of stop without any risk of damaging the bevel.

According to another embodiment variant, the interposing means 12 may comprise a partition 21 resisting to perforation, pervious to the injected or punctured fluids, and being preferably openwork to this end, which divides the chamber 2 into at least a first cavity 22 and a second cavity 23, said first cavity 22 remaining within the reach of the needle 5 whereas the outlet 4 is located in the second cavity 23.

Thus, it is possible to provide the chamber 2 with a sort of double bottom permitting fluid circulation between the cavities 22, 23 but preventing the needle 5 from reaching the outlet 4.

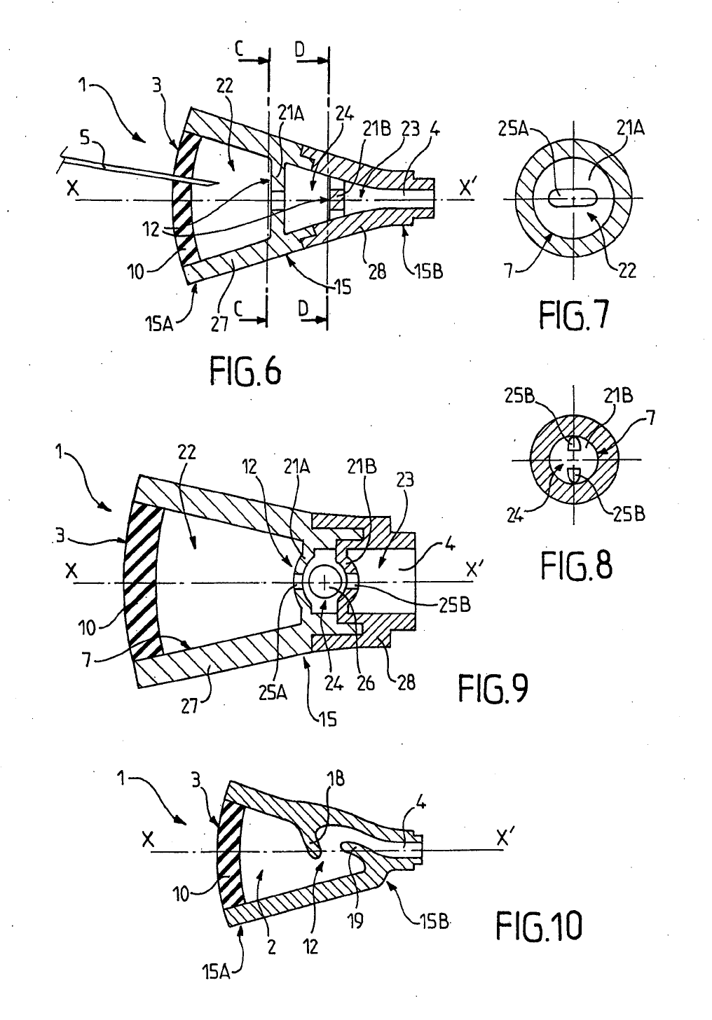

Preferably, as illustrated in FIGS. 6, 7, 8 and 9, the interposing means can be formed by the succession of two openwork partitions 21A, 21B delimiting an intermediate cavity 24 between the first and second cavities 22, 23.

Openings 25A, 25B, for example oblong-shaped, can be arranged in said partitions to enable fluid flowing. Said openings can possibly have a size greater than the diameter of the needle 5. They have then preferably mating shapes and/or a crossed or offset arrangement so as to ensure that the needle 5, even if it is liable to pass through the first partition 21A and to enter the intermediate cavity 24, can not go successively through the two partitions, and thus can not reach the second cavity 23.

According to an embodiment variant illustrated in FIG. 9, the intermediate cavity 24 forms a seat and encloses a ball 26 confined, with a clearance, opposite the opening 25A, said ball 26, possibly flexible, being able to divert or to block the bevel of the needle 5 in case the latter would pass through said opening 25A.

Moreover, as notably illustrated in FIGS. 6 and 9, the truncated cone 15 can advantageously be formed by nesting of two mated tapered sections 27, 28. Advantageously, each of said sections 27, 28 can carry, preferably as a single-piece with the wall thereof, one of the openwork partitions 21A, 21B.

According to an embodiment variant illustrated in FIGS. 11, 13, 15 and 17, the interposing means 12 comprises a curved passageway 30, the curvature of which is sufficiently marked to prevent the needle 5 from passing therethrough.

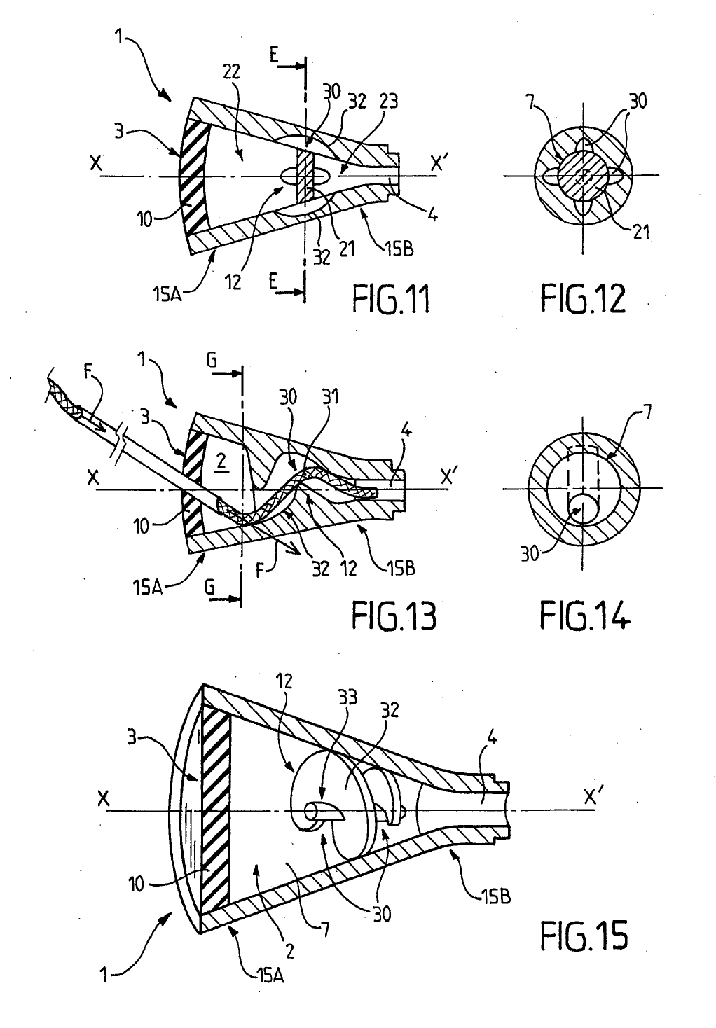

According to an embodiment variant illustrated in FIGS. 11 and 12, the communication between the first and second cavities 22, 23 is ensured by one or more grooves forming a plurality of curved passageways 30, said grooves being arranged fully or in part into the wall 7, at the periphery of the partition 21.

More precisely, according to the invention, the curved passageway 30 is arranged so as to form a sort of meander pervious to fluids but non-piercable by the needle 5, the latter, relatively rigid by nature, being not able to sufficiently deform to conform, from end to end, such a curved relief, so that the bevel remains necessarily captive of said passageway 30, and that, whatever the incidence angle and the point of penetration of said needle 5 at the puncture area 3.

Particularly preferentially, nevertheless, said curved passageway 30, and more generally the interposing means 12, will be shaped so as to permit the passage of a curettage instrument 31, substantially flexible, or even semi-rigid, entering the chamber 2 through the puncture area 3 and intended to pass through the outlet 4 so as to be introduced into the catheter 6.

By “substantially flexible”, it is pointed out that the curettage instrument 31 is flexible enough to support a significant deformation and notably in multiple directions, preferably in an elastic manner, for example by buckling under a thrust force F exerted by the practitioner.

In other words, thought the structural stiffness of said curettage instrument 31 is sufficient to allow progression of the latter within the chamber 2 and through the catheter under the thrust force F, said stiffness is lower than that of the needle 5 and compatible with the passing through of the obstacle formed by the interposing means 12.

Advantageously, the curettage instrument 31 according to the invention will consist of a mandrel to be introduced into a vein, or a metallic wire.

Of course, despite the curved geometry thereof, the curved passageway 30 will keep a sufficient cross-section so as not to form a marked constriction liable to cause significant load losses hindering the flowing of injected and punctured fluids, or else to expose the device 1 to an increased risk of obstruction.

Moreover, the interposing means 12 will be preferably free of wells or others cul-de-sac liable to prematurely block the progression of the curettage instrument 31 by retaining captive the distal end thereof.

In particular, the curved passageway 30 will preferably have at least one guiding ramp 32, having a relatively gentle curvature, liable to direct progressively, by deflexion, the curettage instrument 31 toward the outlet 4.

For example, in an embodiment variant based on that shown in FIG. 11 but according to which the partition 21 would have a face bulged toward the puncture area 3, the guiding ramp 32 could advantageously be formed by the combination of said bulged surface, which would tend to skim the curettage instrument 31 toward the grooves 30, and the bottom of the curved grooves 30 which would then tend to pull down the curettage instrument 31 toward the axis (XX′).

According to a preferential embodiment variant illustrated in FIG. 15, the interposing means 12 comprises, to the above-mentioned ends, a helical element 33 delimiting a spiral curved passageway 30.

Preferably, the axis of said helical element 33 is substantially merged with the longitudinal axis ‘XX’), the entrance of the curved passageway 30 being directed toward the puncture area 3 and the exit of said passageway 30 being opposite the outlet 4.

Preferably, the helical element 33 has at least a complete helical pitch so as to hide the outlet 4 with respect to the puncture area 3. Advantageously, the helix angle (pitch angle) will be chosen so that the wound surface forming the guiding ramp 32 facilitates the progressive flexion of the curettage instrument 31, when the latter passes through the passageway 30, under the driving force F applied by the practitioner.

Thus, in a particularly advantageous manner, it is possible to clean the catheter 6, and notably to unblock the latter in case of obstruction, by introducing the curettage instrument 31 through the puncture area 3 (by means of a cannula or a hollow needle 5, for example), by slipping the former through the curved passageway 30, and through the outlet 4, and through the catheter 6, up to the cluttered area 35. Thus, it is possible to perform a mechanical cleaning of the catheter 6, notably by abrasion, a little as a chimney-sweeping.

Naturally, the present invention is not limited to a curved passageway 30, the curvature of which varies continuously. In particular, said passageway 30 can comprise, for example, a succession of straights sections, juxtaposed to each other at different angles of orientation so as to form a broken line extending in two or three dimensions, the curvature, within the meaning of the invention, being thus obtained in a discrete manner through the different direction changes.

According to the invention, it is possible for the device 1 to be made by assembling only two parts, a septum 10 being added, for example bounded, on a housing 17, or a shell, that is tapered, non-piercable and molded integral with the interposing means 12.

According to another embodiment variant, the device 1 according to the invention is made by modular mounting of four parts, namely: a septum 10, a first tapered section 27 forming a non-piercable rigid shell, for example made of titanium, an elastomeric housing 17 covering said first tapered section 27 and forming the second tapered section 28, and a part forming the interposing means 12, for example a pervious washer 21 forming a partition or an added helical element 33.

The interposing means 12 is then advantageously crimped in the rigid shell or taken in sandwich between the two tapered sections 27, 28.

According to another embodiment variant illustrated in FIG. 17, the device 1 according to the invention comprises the following three parts: a septum 10, a tapered section 27, that is rigid, non-piercable and substantially smooth, and a interposing ring 34 resistant to perforation. Said ring 34 is an extension of the chamber 2, which is preferably cylindrical and with which the interposing means 12 is formed as a single-piece.

Advantageously, said interposing ring 34 is thus placed between the small base of the tapered section 27 and the catheter 6, an end of said ring 34 delimiting the outlet 4 by forming the tip 14 on which said catheter 6 can be added.

Such an arrangement allows to create a possibly flexible, single-piece separation interface between the portion of the chamber 2 within the reach of the needle 5 and the catheter 6, which simplify both the fabrication of the “spare parts” and the assembly of the device 1, respectively of the implantable system 40, that will be described hereinafter as a whole.

Indeed, as illustrated in FIGS. 16 and 17, the device 1 according to the invention is preferably intended to be connected in line with a catheter 6.

It is therefore possible to make up an implantable system 40 comprising a implantable device 1 of the “site” type, as above-described, and also comprising a catheter 6 connected to said device 1 so that the chamber 2 communicates with said catheter 6, and more precisely with the internal duct of the tubing forming said catheter 6, through the outlet 4.

The device 1 can thus advantageously form the tip of an implantable catheter.

It is possible for the device 1 and the catheter 6 to be reversibly assembled so that the system according to the invention could be dismantled.

According to a particularly preferential embodiment variant of the system 40 according to the invention, the device 1 and the catheter 6 are made integral by a positive connection arranged so that they form together a single-piece assembly.

For this reason, it is possible to form said single-piece assembly either during the manufacturing in factory or only during the use in operating block, by irreversibly assembling a device 1 and a catheter 6 provided separately.

Notably, the catheter 6 will be slipped onto the tip 14 and attached to the latter by bonding.

Moreover, it is possible that the device 1 forms a tip intimately integral with the catheter 6, or even that at least one part of the device 1 is formed as a single-part with the tubing of said catheter 6 and forms a bulge of the end 6A. Thus, it is possible to make a substantially continuous junction having no protrusion and no abrupt shape liable to offer a hold to tissue coating or else to damage the surrounding tissues.

Thus, in a particularly advantageous manner, the device 1 according to the invention is particularly compact and has an atraumatic shape that facilitates the implantation and improves both the physical and the aesthetic comfort for the patient.

Advantageously, the device 1 according to the invention has a particularly simple structure, requiring a few parts and assembling operations, which allows to limit considerably the fabrication cost thereof.

Moreover, the implantable site according to the invention is particularly ergonomic and allows the practitioner to perform an intuitive pricking because the latter is performed substantially in the same manner as in a natural vein.

Advantageously, the device 1 according to the invention combines a useful pricking surface, which is particularly extensive and easily locatable, with quite reduced whole bulkiness.

In a particularly advantageous manner, the device 1 according to the invention allows a safe implementation of the pricking operation, insofar as it allows, on the one hand, to easily identify and locate the puncture area, and on the other hand, to respect the physical integrity of the implantable site as well as the catheter and the needle.

Finally, the possibility to maintain on a regular basis the system 40 according to the invention, and more precisely the catheter 6, by means of a curettage instrument, advantageously allows to optimize the service life of said system 40 following its implantation.

### INDUSTRIAL APPLICABILITY

The invention finds its industrial application in designing and making implantable sites for injecting and/or drawing fluids.

1. An implantable device (1) for injecting and/or drawing fluid into or from a human or animal organism, said device comprising a chamber (2) having a puncture area (3), adapted to be capable of being pierced by a needle (5) for the purpose of injecting and/or drawing a fluid, and an outlet (4) located opposite said puncture area (3) and intended to connect said chamber (2) with a duct such as a catheter (6), said chamber (2) being so shaped to receive the needle (5), said device (1) having a substantially elongated shape along a longitudinal axis (XX′), in that the puncture area (3), the chamber (2) and the outlet (4) are substantially aligned in the direction of said longitudinal axis (XX′), and in that it comprises an interposing means (12) projecting into the chamber (2) so as to prevent the needle (5) that enters said chamber (2) through the puncture area (3) from reaching the outlet (4).

2. The device according to claim 1, in which said device is shaped so that it can be connected in line with a catheter (6), the puncture area (3), the chamber (2), the outlet (4) and the end (6A) of the catheter (6) that is adjacent to said outlet (4) being then preferably substantially aligned along the longitudinal axis (XX′).

3. The device according to claim 1 in which said device has the form of a truncated cone (15) whose base (15A) substantially corresponds to the puncture area (3) and apex (15B) substantially corresponds to the outlet (4).

4. The device according to claim 1 in which the chamber (2) has a flared shape, preferably a truncated shape, said chamber (2) widening between the outlet (4) and the puncture area (3).

5. The device according to claim 1 in which the chamber (2) is delimited by a wall (7) and in that said wall (7) is adapted to resist to perforation by the needle (5), at least in the space of the chamber (2) that is within the reach of said needle (5).

6. The device according to claim 5, in which only the fraction of the wall (7) that delimits the space of the chamber (2) within the reach of the needle (5) is both stiffened and reinforced against perforations, preferably by means of a single shell element, the rest of the wall (7) being made of an elastomeric material such as silicone or PU.

7. The device according to claim 5 in which the interposing means (12) comprises at least one fixed element with respect to the wall (7) and/or with respect to the outlet (4) and/or with respect to the puncture area (3).

8. The device according to claim 5 in which the interposing means (12) comprises one or more excrescencies (16) projecting from the wall (7) toward the inside of the chamber (2).

9. The device according to claim 8, in which said excrescencies (16) are formed as a single-piece with the wall (7).

10. The device according to claim 1 in which said interposing means (12) comprises a plate (18) forming a baffle.

11. The device according to claim 10, in which said the interposing means (12) comprises a plurality of plates (18, 19) stepped in baffle along the longitudinal axis (XX′).

12. The device according to claim 11, in which said plates (18, 19) overlap each other so that the respective projections thereof onto a plane perpendicular to the longitudinal axis (XX′) cut together.

13. The device according to claim 1 in which said interposing means (12) comprises a partition (21) resisting to perforation, pervious to the injected or punctured fluids, which divides the chamber (2) into at least a first cavity (22) and a second cavity (23), said first cavity (22) remaining within the reach of the needle (5) whereas the outlet (4) is located in the second cavity (23).

14. The device according to claim 1 in which said interposing means (12) comprises flexible elements capable of mechanically resisting to the progression of the needle (5), without damaging the bevel of said needle.

15. The device according to claim 14, in which said interposing means (12) comprises a damping element (20) forming an elastic stop for the bevel of the needle (5), said damping element (20) being capable of deforming so as to progressively block the progression of said needle (5).

16. The device according to claim 1 in which said device has a rotational geometry about the axis (XX′).

17. The device according to claim 5 in which said wall (7) comprises an elastomeric housing (17) of the silicone type.

18. The device according to claim 1 in which said interposing means (12) comprises a curved passageway (30), the curvature of which is sufficiently marked to prevent the needle (5) from passing therethrough.

19. The device according to claim 1 in which said interposing means (12) comprises a helical element (33) delimiting a spiral curved passageway (30).

20. The device according to claim 1 in which said interposing means (12) is shaped so as to permit the passage of a substantially flexible curettage instrument (31) entering the chamber (2) through the puncture area (3) and intended to pass through the outlet (4) so as to be introduced into the catheter (6).

21. The device according to claim 1 in which said puncture area (3) comprises a self-sealing membrane (10).

22. An implantable system (40) comprising an implantable device (1) according to claim 1 and said system (40) having further a catheter (6) connected to said device (1) so that the chamber (2) communicates with said catheter (6) through the outlet (4).

23. The system (40) according to claim 22, in which said implantable device (1) and the catheter (6) are made integral by a positive connection arranged so that they form together a single-piece assembly.
